(19) United States
(12) Patent Application Publication
(10) Pub. No.: US 2005/0036996 A1
(43) Pub. Date: Feb. 17, 2005

(54) Absorbable composition containing propionic bacteria capable of releasing nitric oxide in the human or animal alimentary canal

(76) Inventors: Edmond Roussel, Avenay (FR); Charles Legrand, Caen (FR); Marc LeGrand, Caen (FR); Nathalie Roland, Rennes (FR)

Correspondence Address: BAKER & DANIELS;111 E. WAYNE STREET, SUITE 800, FORT WAYNE, IN, 46802, US

(21) Appl. No.: 10/946,156

(22) Filed: Sep. 21, 2004

Related U.S. Application Data
(63) Continuation of application No. 09/331,554, filed on Aug. 23, 1999, now abandoned.

(30) Foreign Application Priority Data
Jan. 28, 1997 (FR) 97/00885
Dec. 24, 1996 (FR) 96/15977

(51) Int. Cl. A61K 45/00; C12P 7/60

(52) U.S. Cl. 424/93.45

(57) ABSTRACT

The invention concerns the use of propionic bacteria for obtaining a current feeding composition or an absorbable dietetic or medicinal composition capable of releasing physiologically significant amounts of nitric oxide in the human or animal alimentary canal.

The present invention relates to an absorbable common food composition or an absorbable dietary or medicinal composition containing propionibacteria which are capable of releasing physiologically significant amounts of nitric oxide into the human or animal digestive tract.

For many decades, it has been entirely ignored that nitric oxide is one of the elements necessary for life and for maintaining life; consequently, up until 4 or 5 years ago, researchers paid little attention to the benefits associated with the presence of this oxide, either in medicine, in nutrition or in physiology.

It is only very recently that an impressive number of physiological functions have been attributed to nitric oxide and that the hypothesis was put forward that this gas might be involved extensively in functions as diverse as controlling arterial pressure, the non-specific cytotoxic function of macrophages, platelet aggregation and neurotransmission, or controlling the motility of the digestive tract.

Starting with this assumption, research related to nitric oxide mushroomed and the importance of this gas was able to be confirmed.

It is known that nitric oxide, which is a very unstable gas (half-life of less than 5 seconds in biological systems), is produced by biosynthesis in the human or animal body from L-arginine by a group of enzymes known as the NO-synthases (NOS), of which two main types exist, namely, on the one hand, the constituent NOSs which are expressed in particular in the endothelial cells, the blood platelets and the neurons, and, on the other hand, the inducible NOSs which are expressed mainly by certain cells of the immune system (macrophages and polymorphonuclear leukocytes in particular), by vascular smooth muscle and endothelial cells.

It should be noted that the production of NO by the inducible NOSs is greater than the production of NO by the constituent NOSs by several orders of magnitude, but that, in any case, this production remains relatively small.

Now, given the abovementioned beneficial role of nitric oxide, it would be desirable to be able to increase this production in particular by using the natural route of food metabolism.

However, no means for achieving this result have ever been proposed hitherto.

The object of the invention is to fill in this absence.

In accordance with the invention, it has been possible to achieve the desired aim by observing that, surprisingly, bacteria of one specific type, the propionibacteria, are capable of producing nitric oxide, and that, among these bacteria, certain species and certain strains among these species produce it in large amounts.

They are propionibacteria which, although not belonging to the group of lactic bacteria or bifidobacteria conventionally introduced into the body via milk-based desserts or other fermented dairy products, have nevertheless been present in human food for centuries: indeed, it is these bacteria which produce the holes during the manufacture of the cheese known as “emmental” which, after maturing, contains about 10⁹ cells/g of propionibacteria.

It should be noted that the fermentation of these bacteria produces, inter alia, propionic acid, acetic acid and carbon dioxide.

The abovementioned finding is all the more surprising since it has been possible to confirm that lactic acid bacteria, bifidobacteria and/or yeasts, commonly used in the agrifood sector, do not produce carbon monoxide.

The invention consequently relates to the use of propionibacteria to produce an absorbable common food composition or an absorbable dietary or medicinal composition which is capable of releasing physiologically significant amounts of nitric oxide into the human or animal digestive tract.

In accordance with the invention, this composition can consist of an elaborate preparation and/or can be presented in liquid form (in particular a fermented liquid), in dehydrated form or in a form of intermediate moisture content.

More specifically, it should be noted that, without, however, departing from the scope of the invention, the composition can be:
either in the form of a specific preparation justified by its sole physiological purpose, namely the ingestion of propionibacteria capable of releasing physiologically significant amounts of nitric oxide,
or in the form of an elaborate food preparation which, in parallel, has another more strictly energetic or functional purpose; in the latter case, the propionibacteria can be added or incorporated into the foods themselves, in particular into cheeses, into dietary fibre such as cereal flakes, or into fermented milks, dessert creams, cakes and/or tonic drinks, etc.

In accordance with the invention, the propionibacteria can be introduced in the form of a biomass or in the form of a leaven capable of multiplying in situ.

When it is dehydrated, the composition is advantageously in the form of individual fractions containing the dose of bacteria which needs to be regularly absorbed.

These fractions can be ingested directly or prediluted in a liquid; they can be packaged in a form which facilitates absorption: tablets, sachets of granulated powder, liquid, etc.

It has been confirmed that such concentrated dehydrated preparations of propionibacteria stored for one year at ±4° C. undergo a fall in concentration of less than one Log unit.

Experience has shown that gelatin capsules, which may or may not be gastroresistant, are a particularly advantageous type of packaging.

According to another characteristic of the invention, each individual fraction contains a large number of bacteria, preferably more than 10⁹ bacteria.

Various experiments (summarized below) confirmed the quite specific ability of different strains of propionibacteria to produce NO during their culturing, firstly indirectly by measuring the nitrite ions NO₂ [sic], and then directly by mass spectrometry analysis in anaerobic medium.

During these experiments, the nitrite concentration in arginine-rich and nitrate-poor (50 μM) media was studied, in a first stage, and it was realized that arginine is not a determining factor in the observed production of NO.

In a second stage, nitrate-supplemented media were investigated. The results obtained in the latter case revealed the nitrate-dependent nature of the production of nitric oxide.

1—Comparative Preliminary Tests

Various bacterial strains (yoghurt inoculum, bifidobacteria, lactobacillus) were cultured in the presence of a reconstituted milk medium (100 ml) supplemented with a yeast extract (10 g/l) and then incubated at 37° C.

The accumulation of nitrite was measured over time.

These preliminary tests were carried out under the following conditions:
incubation at 37° C. for 0, 4, 7 or 10 hours,
3 repetitions
assay of the nitrites by the Bran-Luebbe system.

On account of the nature of the extracts to be analyzed, a step of purification of the samples was subsequently carried out by a double centrifugation (2×10 min., 4° C., 15,000 rpm), followed by an ultrafiltration on a Miniprep 10 cartridge (retention of the proteins of MW>10 kD) and then partial purification by passing the sample through Waters C18 resin (55-105 μm).

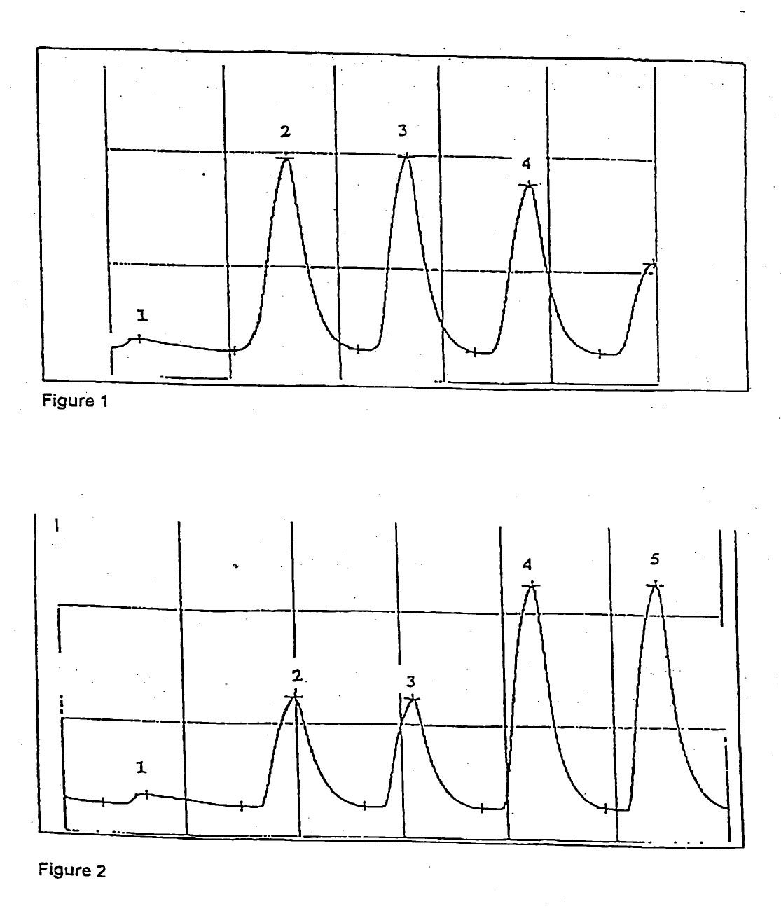

This method was tested, in a first stage, on standard nitrite samples (FIG. 1), and then on Lactobacillus culture extracts incubated for 7 hours, to which a known amount of nitrite was added or otherwise (FIG. 2).

FIG. 1 represents the calorimetric profiles obtained on a Bran-Luebbe automatic analysis line:
(1) for a bifidobacteria culture medium after incubation for 10 hours,
(2) for a standard nitrite solution,
(3) for this same, ultrafiltered solution,
(4) for this same solution, ultrafiltered and passed through C18 resin.

FIG. 2 represents the colorimetric profiles obtained on a Bran-Luebbe automatic analysis line:
(1) for a Lactobacillus culture medium after incubation for 10 hours at 37° C.,
(2) (3) for a standard solution containing 410 μg of nitrite/l,
(4) (5) for a Lactobacillus culture medium after incubation for 10 hours at 37° C., to which was added a known amount of nitrite in order to obtain a solution containing 820 μg/l of nitrite.

These samples were purified by centrifugation-ultrafiltration and passage through C18 resin under the conditions described above.

In accordance with these tests, no accumulation of nitrite could be detected, either using yoghurt inoculum, bifidobacteria or Lactobacillus, irrespective of the incubation time (0, 4, 7 or 10 hours).

2—Demonstration of the Accumulation of Nitrite by Propionibacterium Cultures

The possible presence of nitrate or nitrite in the preparation of the YEL medium was investigated beforehand by calorimetric assay (Boehringer kit): it was thus possible to demonstrate the presence of an appreciable amount of nitrate in this medium (concentration of about 50 to 100 μM) which might come from the yeast extract used for the manufacture of this medium; on the other hand, it was confirmed that the YEL medium was totally free of nitrite.

Propionibacterium cultures (1 g of lyophilisate per 100 ml of YEL medium) were tested.

These tests were carried out under the following conditions:
incubation at 30° C. for 24, 48 or 72 hours,
3 repetitions for the 24-hour incubation,
stopping the incubation by boiling,
purification of the product by centrifugation and passing the extract through C18 resin,
assaying the nitrites in the medium by analysis on the Bran-Luebbe system.

The nitrites accumulated by the propionibacteria were assayed in order to establish kinetics of nitrite accumulation as a function of the incubation time of the bacteria on YEL medium.

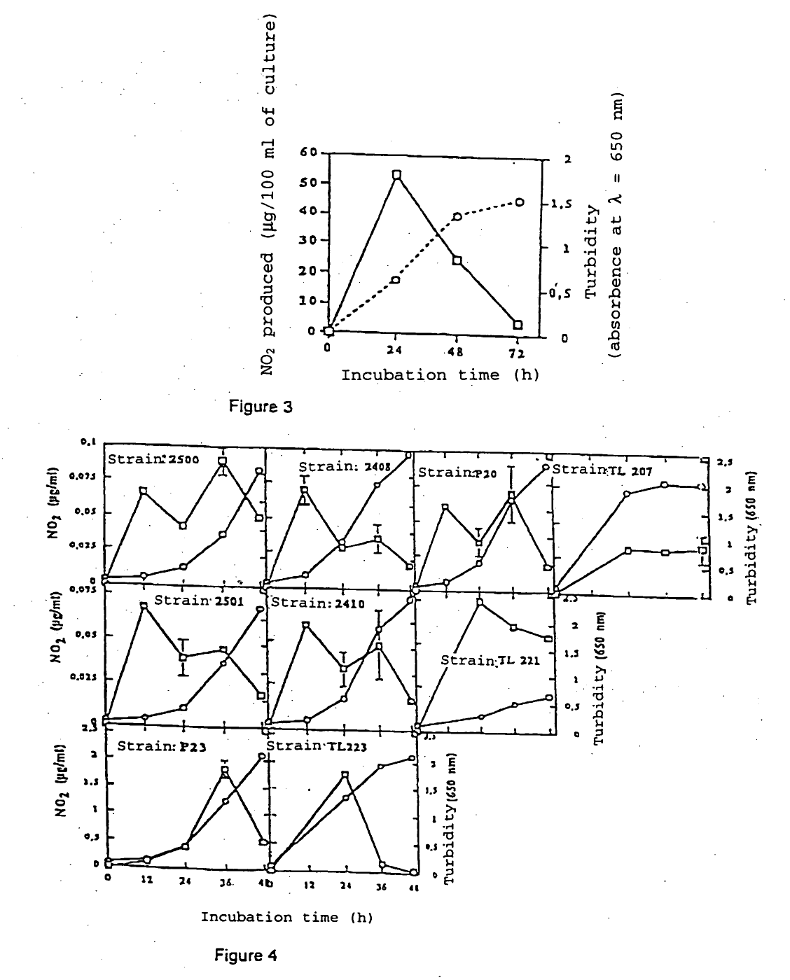

FIG. 3 represents, on the one hand, the variations in the amount of nitrite produced (in μg/100 ml of culture) as a function of the incubation time (in hours) (□) and, on the other hand, the variations in the turbidity (absorbence at λ=650 nm) also as a function of the incubation time (◯).

This figure shows that the amount of nitrite is at a maximum at 24 hours and then decreases significantly after 48 and 72 hours of incubation.

It may reasonably be considered that this fall results from the reduction of the nitrite to NO, N₂O or other compounds by nitrite reductase.

In accordance with the invention, it was possible to prove that the accumulation of NO₂ depends on the propionibacterium species or strains used.

This situation was confirmed by the tests summarized below:

3—Demonstration and Comparison of the Nitrite Accumulations in the Culture Medium in the Case of 9 Strains of 4 Different Propionibacterium Species

In accordance with this test, the strains P20, P23, 2408, 2410, 2500 and 2501 of the species P. freudenreichii and the strains TL221, TL223 and TL207 belonging, respectively, to the species P. thoenii, P. acidipropionici and P. jensenii, were studied.

It should be noted that the TL (technologie laitiére [dairy technology]) strains are strains belonging to the INRA-LRTL, while the strain P23 (or ITG23) was registered at the Collection Nationale des Cultures de Micro-organismes [National Collection of Microorganism Cultures] (CNCM) of the Institut Pasteur under the number I-1804 dated 18.12.96.

The various propionibacterium strains (1 g of lyophilisate or 5 ml of fresh culture) were cultured on 100 ml of YEL medium containing about 50 μm of nitrate, under the following conditions:
incubation at 30° C. for 12, 24, 36 or 48 hours,
3 repetitions,
stopping the incubation by boiling,
purification of the product by centrifugation and passing the extract through C18 resin,
accumulation of nitrite in the medium, measured by analysis on the Bran-Luebbe system,
estimation of the fermentation of each culture measured by reading the absorbence at 650 nm.

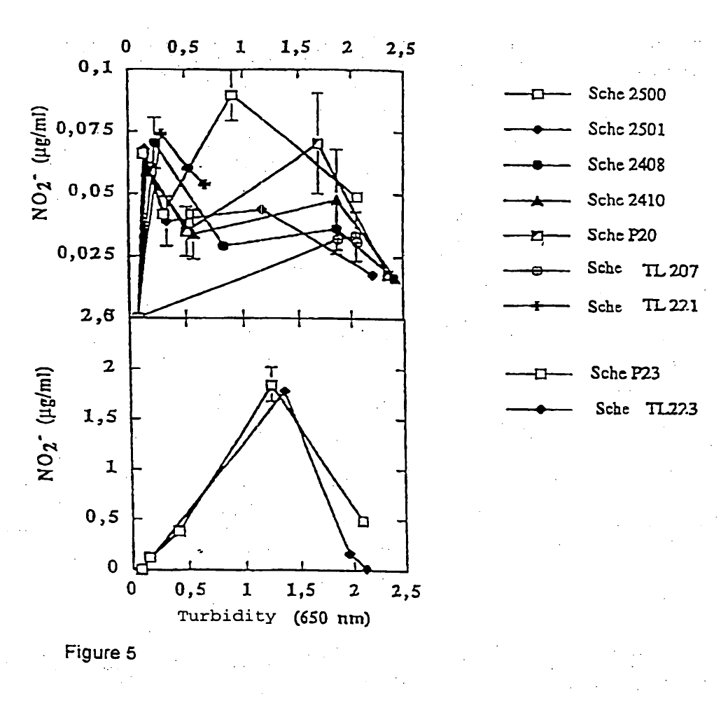

The results obtained for each strain are collated in FIG. 4.

This represents, in each case, the variations in the accumulation of nitrite (□) and in the turbidity of the culture medium (◯) as a function of the incubation time. Each value corresponds to the average±the standard error of mean for n=3.

It should be noted that the scales of nitrite accumulation are 25 times greater in the case of the strains P23 and TL223.

These results prove that the bacterial growth, estimated from the change in turbidity of the culture medium, is similar for all of the strains studied, reaching about 2 to 2.5 OD after incubation for 48 hours, except for the strain TL221 for which the turbidity reaches only 0.6 OD after 2 days.

On the other hand, highly significant differences exist as regards the accumulation of nitrite as a function of time.

Specifically, the strains 2500, 2408, P20, 2501, 2410, TL207 and TL221 accumulate a relatively small amounts of nitrite, the maximum (0.1 μg of NO₂⁻/ml) being reached after incubation for 36, 12, 36, 12, 12, 24 and 24 hours of incubation, respectively.

In contrast, a much greater accumulation of nitrite was obtained with strains P23 and TL223, which accumulate, at the maximum, 1.8 μg of NO₂⁻/ml after 36 and 24 hours of incubation, respectively.

It should be noted that the propionibacterium strain analyzed in the abovementioned second test (FIG. 3) had an intermediate position with a maximum NO₂⁻ accumulation of about 0.5 μg/ml.

These tests thus made it possible to state that significant differences exist between the amounts of nitrite which can be produced by different propionibacterium strains from four different species, these differences being independent of the growth of these strains.

These results could be confirmed by studying the change in the nitrite concentration of the culture medium as a function of its turbidity and thus, approximately, of the bacterial growth, for each strain.

The results of these last tests are given in FIG. 5, in which each value corresponds to the average±standard error of mean for n=3.

The abovementioned tests were designed to establish that, among the strains studied, the strain TL223 accumulates nitrites to the greatest extent, these nitrites disappearing after 12 hours. This strain was thus selected in the context of complementary tests relating to the direct measurement of the production of nitric oxide by mass spectrometry analysis in anaerobic medium.

4—Preliminary Measurement of the Production of NO by the Strain TL223 Under a Helium Atmosphere

In accordance with these tests, the cultures were prepared in 10 ml tubes containing 5 ml of YEL medium containing about 50 μM of nitrate and 0.25 ml of fresh culture of the strain TL223.

The atmosphere of the tubes was immediately evacuated by a flow of helium (100 ml/min) for 100 seconds.

The accumulation of NO in the atmosphere of the tubes was then measured over time under the following conditions:
incubation at 30° C. for 24, 48 or 72 hours,
4 repetitions,
measurement of the accumulation of NO by mass spectrometry analysis,
estimation of the fermentation of each culture, measured by reading the absorbence at 650 nm.

During a preliminary test, the gas purification system (Roboprep G+)—mass spectrometer (Twenty-Twenty) was calibrated with increasing amounts of nitric oxide.

This gas was generated from NaNO₂ in the presence of a solution of KI and H₂SO₄.

The identification and quantification of the nitric oxide were performed on the basis of its mass: 30 for ¹⁴N¹⁶O and 31 for ¹⁵N¹⁶O and ¹⁴N⁷O. This identification was then confirmed by measuring the isotope ratio: 31/30=[¹⁵N¹⁶O+¹⁴N¹⁷O]/¹⁴N¹⁶O. It should be noted that the theoretical isotope ratio 31/30 for NO is 0.00367 in the absence of contamination with ¹⁷O.

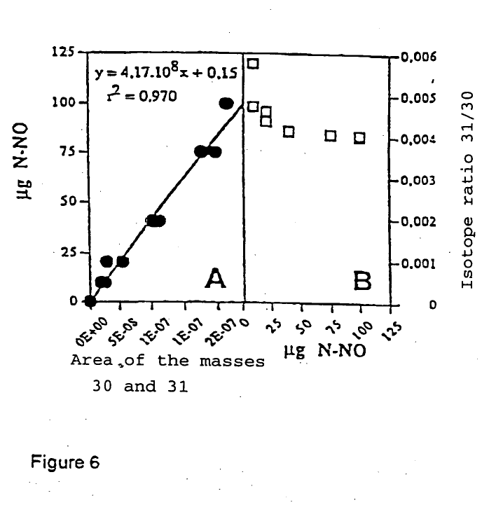

The results of this preliminary test are given in FIG. 6, in which the left-hand part (A) corresponds to the calibration curve of the mass spectrometer used to quantify the nitric oxide, while the right-hand part (B) corresponds to the measurement of the isotope ratio 31/30.

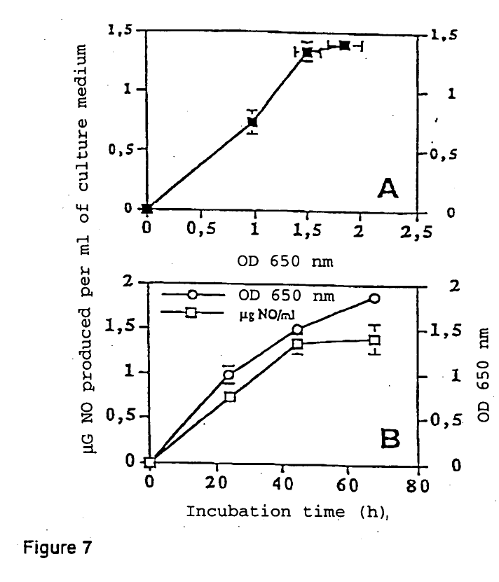

The actual results of this test are given in FIG. 7.

More specifically, FIG. 7A represents the variations in the accumulation of NO in the atmosphere of the tubes as a function of the turbidity of the medium, while FIG. 7B represents the variations in this accumulation as a function of the incubation time.

The vertical or horizontal bars indicate, when they are wider than the symbol, the standard error of mean for n=4.

A comparison of FIG. 7B (which represents the change over time in the turbidity of the culture medium under helium) with FIG. 4 (which represents this same change in air) shows that the growth of the strain TL223 is not significantly affected by an atmosphere consisting essentially of helium.

It was also possible to establish that the rate of accumulation of nitric oxide in the atmosphere is constant over about the first 45 hours of incubation, and then curves off (FIG. 7B), which corresponds to a turbidity close to 1.5 OD (FIG. 7A).

After incubation for about 65 hours (turbidity greater than 1.7 OD), about 1.5 μg of NO are accumulated in the helium atmosphere per 1 ml of culture medium.

The order of magnitude obtained is compatible with the nitrite contents measured in the medium of cultures in contact with air.

In the latter case, it was in fact found (FIG. 5) that the strain TL223 accumulated a maximum 1.8 μg of NO₂⁻/ml for a turbidity of 1.5 OD, which corresponds to about 1.2 μg of NO/ml.

From this preliminary direct measurement of the production of NO by the strain TL223, an attempt was made, in accordance with the invention, to confirm the route of synthesis of this nitric oxide, and the notion of investigating whether or not this synthesis is stimulated by a supply of nitrite or nitrate was proposed for this purpose.

Such a stimulation was able to be confirmed by means of the tests summarized below:

5—Study of the Stimulation of the Production of NO by Supplying Nitrite or Nitrate

With the aim of investigating whether or not the production of NO by propionibacteria is possible from NO₂⁻ or NO₃⁻, an investigation to see whether or not an increase in the production of NO by the strain TL223 when it is in the presence of 1 mM KNO₂ or KNO₃ (with and without isotopic labeling with ¹⁵N) is observed was carried out.

This experiment was carried out under the following conditions:
the strain TL223 was inoculated at a concentration of 1% in a YEL medium alone (control) or with addition of 1 mM KNO₂, KNO₃ or K¹⁵NO₃ with isotopic labeling with ¹⁵N (at a concentration of 50%),
incubation at 30° C. for 24, 48 and 72 hours,
3 repetitions per point and per treatment,
accumulation of the NO and determination of the isotope ratio by mass spectrometry,
estimation of the fermentation of each culture (turbidity) measured by reading the absorbence at 650 nm,
assay of the nitrate and the nitrite after recovery of the bacterial media, centrifugation and measurement with a Boehringer kit.

The accumulation of NO as a function of the NO₃⁻ concentration (100, 150, 350, 650 and 1050 μM) or of the NO₂⁻ concentration (50, 100, 400, 800, 1000 μM) after incubation for 72 hours at 30° C. (TL223 inoculated at a concentration of 1% in a YEL medium) was also analyzed.

The samples to be analyzed were distributed in NO analysis tubes whose atmosphere was immediately evacuated by flushing with helium for 150 s in order to obtain strict anaerobic conditions.

The effect of flushing with helium on a possible detection of NO by the mass spectrometer was tested on a sterile YEL medium: this YEL medium, preflushed with helium, was incubated at 30° C. for 72 hours. After 0, 24, 48 and 72 hours of incubation (3 repetitions), no production of NO was detected and the turbidity values remained at 0.

It was thus possible to check the quality of the helium flush, the absence of any bacterial contamination and the absence of interaction between the helium flush and the measurement of NO by mass spectrometry.

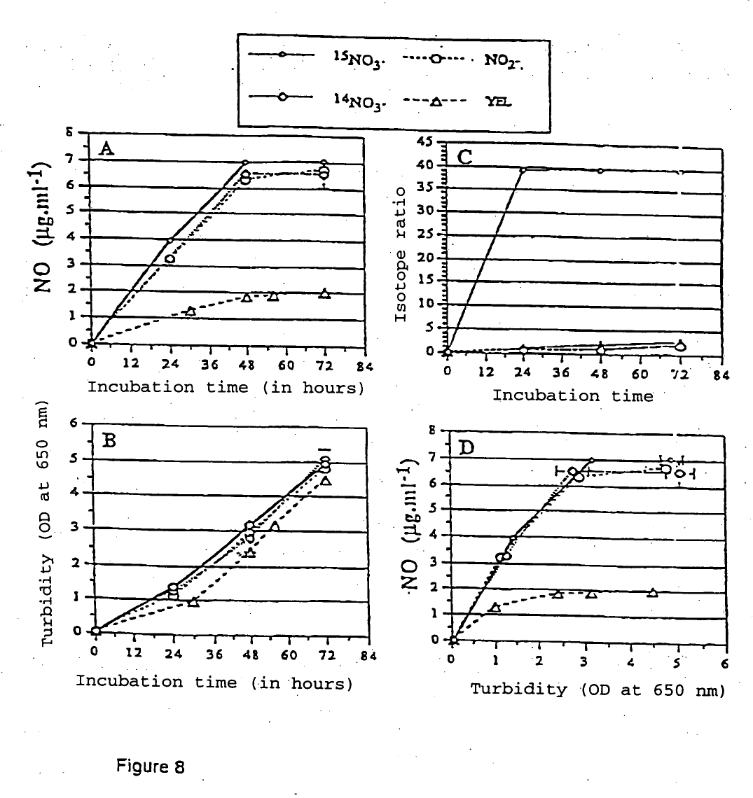

The abovementioned tests gave the results reported in FIG. 8, as regards the kinetics of accumulation of NO in the presence of nitrite or nitrate.

More specifically:

FIG. 8A represents the variations in the accumulation of NO as a function of time,

FIG. 8B represents the variations in the turbidity as a function of time,

FIG. 8C represents the variations in the isotope ratio [mass 31/(masses 30+31)] as a function of time,

FIG. 8D represents the variations in the production of NO as a function of the turbidity.

Each of these figures relates to the strain TL223 cultured on a YEL medium alone, in the presence of 1 mM nitrate (¹⁵NO₃⁻—labeled to a proportion of 50% and ¹⁴NO₃⁻) and in the presence of 1 mM nitrite.

The vertical bars represent the ±standard error of mean for n=3 when they are larger than the symbol.

These figures prove that the accumulation of NO by TL223 cultured on 1 mM KNO₃ (labeled with nitrogen-15 or unlabeled) or on 1 mM KNO₂ is close to 7 μg of NO/ml after incubation for 48 hours at 30° C.; this value is 3.5 times greater than the production of NO in the case of a YEL medium not supplemented with nitrate or nitrite (FIG. 8A). These differences are not due to growth variations generated by the composition of the medium, since the turbidity at the end of growth is of the same order of magnitude on a YEL medium alone (4.5 OD units after 72 hours) and on the same medium supplemented with nitrate or nitrite (about 5 OD units after 72 hours —FIGS. 8B and 8D).

FIG. 8C reveals that supplying K⁵NO₃ labeled to a proportion of 50% makes it possible to obtain NO containing labeling in a proportion of about 40% after incubation for 48 hours: nitrogen of mass 15 supplied in the form of nitrate is thus found in the NO synthesized by the strain TL223.

It was also observed that when the strain TL223 is cultured on K¹⁵NO₃, the profile of the peak of mass 31 (¹⁵N¹⁶O) increases to a great extent relative to the NO analyzed on a YEL medium containing unlabeled nitrate, thereby confirming this situation.

Furthermore, the addition of 1 mM unlabeled KNO₃ or KNO₂ leads to a production of NO with an isotope ratio of 0.75% (incubation for 48 hours—FIG. 8C), which is very close to the values for the natural isotope ratio of NO (about 0.4%).

These last observations very clearly confirm that the gas analyzed by mass spectrometry was indeed NO.

These tests thus made it possible to state that the strain TL223 is capable of synthesizing NO directly from nitrite or in the presence of nitrate after reducing this nitrate to nitrite.

On the basis of the isotope ratio values obtained on YEL medium supplemented with 1 mM K¹⁵NO₃ labeled to a proportion of 50%, it is possible to deduce the degree of conversion of nitrate into nitrite: thus, about 20% of the K¹⁵NO₃ supplied is converted into nitric oxide by the strain TL223.

The nitrate, initially present in the sterile YEL medium, explains the observed NO production by TL223 (of about 2 μg of NO/ml after incubation for 72 hours—FIG. 7).

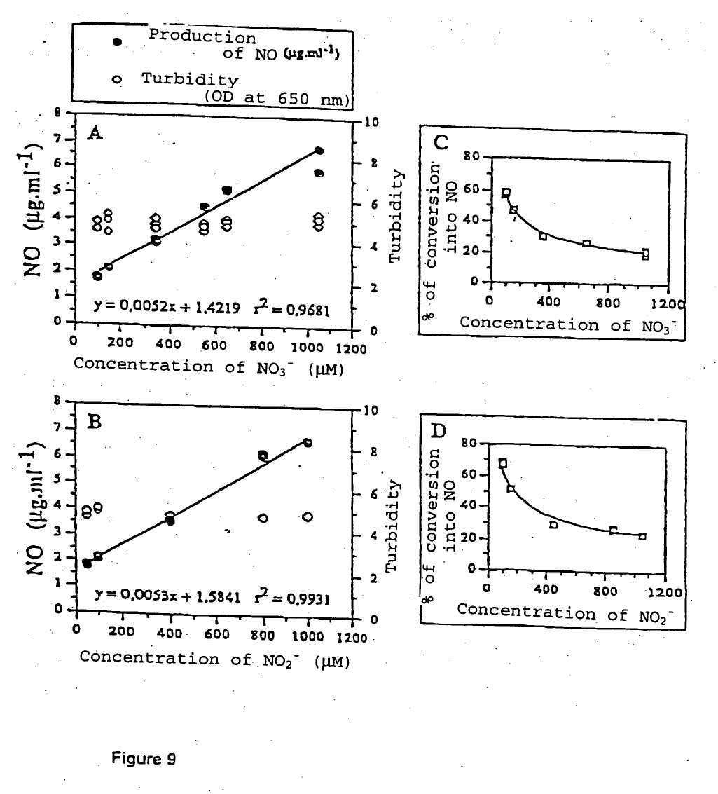

The variations in NO production as a function of the nitrite or nitrate concentration are represented in FIG. 9. More specifically:

FIG. 9A represents the variations in NO production and the change in the turbidity as a function of the initial nitrate concentration, by the strain TL223 cultured on YEL medium after incubation for 72 hours,

FIG. 9B represents the variations in NO production and the change in the turbidity as a function of the initial nitrite concentration, by the strain TL223 cultured on YEL medium after incubation for 72 hours,

FIG. 9C represents the variations in the degree of conversion of nitrate into NO, as a function of the initial nitrate concentration,

FIG. 9D represents the variations in the degree of conversion of nitrite into NO, as a function of the initial nitrite concentration.

In these figures, the nitrate concentrations have been corrected taking into account the presence of about 50 μM nitrate in the YEL medium alone, and are as follows: 100, 150, 350, 550, 650 and 1050 μM.

The nitrite concentrations are as follows: 50, 100, 400, 800 and 1000 μM.

These figures show that, for the ranges chosen, the production of nitric oxide by the strain TL223 is proportional to the initial concentration of nitrate (FIG. 9A) or of nitrite (FIG. 9B) in the YEL medium. This relationship is linear.

In both cases, no plateau phase was observed, which leads to the assumption that the nitrate or nitrite concentrations used do not make it possible to obtain the maximum level of accumulation of NO by the propionibacterium TL223.

It should also be noted that the presence of high concentrations of nitrate or nitrite do not affect the bacterial growth, given that the turbidity values at 72 hours are very similar for all the concentrations which were tested.

It should moreover be pointed out that the curves obtained in FIGS. 9A and 9B can be superimposed, which proves that the NO produced comes directly from the nitrite or nitrate via reduction of the latter to nitrite.

Furthermore, these results show that, with the strain TL223, the step of reduction of nitrate to nitrite is not limiting for the nitrate concentrations chosen in this experiment.

It is also important to point out that the degree of conversion of the NO₃— (FIG. 9C) and of the NO₂⁻ (FIG. 9D) changes as a function of the amount of substrate available, passing from 20 to 60% when the NO₃⁻ or NO₂⁻ concentration passes from 1000 to 100 μM.

This suggests that the production of NO is strongly regulated and that it is predominant over the use of nitric nitrogen for the nitrogenous syntheses.

From the abovementioned conclusions, the production of NO was studied in 12 strains of propionibacteria of two different species. The results of these tests are summarized below:

6—Study of the Production of NO by Different Propionibacterium Strains

In accordance with this test, the propionibacterium strains selected were the following:
P. freudenreichii: LS410, LS2501, LS2502, ITG 23, CNRZ89, CNRZ277, CNRZ81.
P. acidipropionici: TL223, NCDO1072, PR75, CNRZ80, CNRZ86, CNRZ287.

It should be noted that the strains CNRZ80, CNRZ81, CNRZ86, CNRZ89, CNRZ277 and CNRZ287 belong to the INRA-CNRZ public collection, while the strain NCDO1072 belongs to the British collection “National Collection of Dairy Organisms”; the other strains belong to private collections.

After inoculation on YEL medium in the presence of 550 μM nitrate, the bacterial cultures were immediately distributed in 5 ml units in sealed tubes and flushed with helium (anaerobic conditions). Next, the propionibacterium strains were subjected to the following experimental conditions:
incubation at 30° C. for 24, 48 and 72 hours,
3 repetitions,
accumulation of NO in the atmosphere and determination of the isotope ratio by mass spectrometry,
estimation of the fermentation of each culture, measured by reading the absorbence at 650 nm,
assay of the nitrate and nitrite after recovering the bacterial media, centrifugation and measurement with a Boehringer kit.

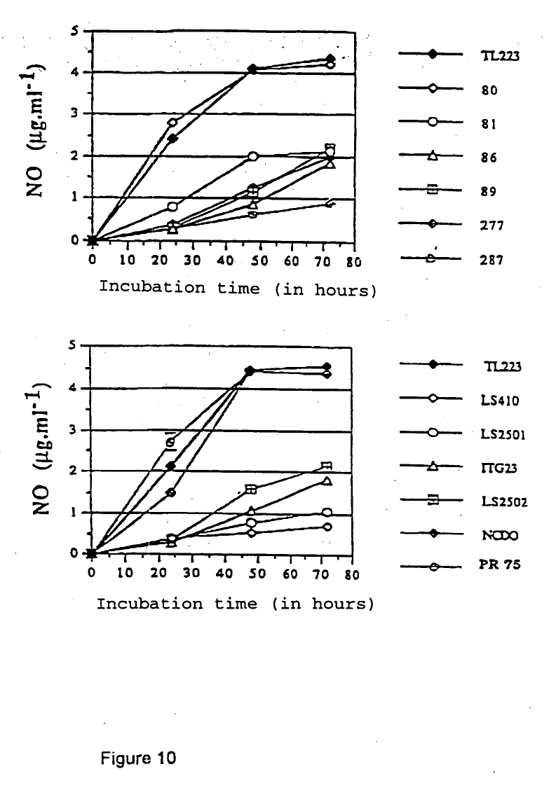

FIG. 10 represents the variations in the accumulation of NO as a function of time by the strain TL223 and the 12 other propionibacterium strains, cultured on a YEL medium in the presence of 550 μM nitrate. The strain TL223 was represented in each case for comparative purposes.

The vertical bars represent the ±standard error of mean for n=3 when they are larger than the symbol.

This figure shows the existence of large divergence between the propionibacterium strains.

Globally, by comparing the levels of production of NO after incubation for 72 hours, the strains can be classified in three categories:
strains capable of producing from 4 to 4.5 μg of NO/ml: TL223, CNRZ80, NCDO1072 and PR75. The isotope ratio for the NO produced by these strains is between 2 and 2.5% (T=72 h),
strains capable of producing about 2 μg of NO/ml: CNRZ81, CNRZ86, CNRZ89, CNRZ277, LS2502 and ITG23. The isotope ratio for the NO produced by these strains is between 4 and 5.5% (T=72 h),
strains producing less than 1 μg of NO/ml: LS410, LS2501 and CNRZ287. Despite the presence of 550 μM nitrate in the culture medium, these 3 strains produced only very small amounts of NO and the isotope ratio was about 10 to 13% (T=72 h). These values suggest that the peaks of masses 30 and 31 detected in these bacteria might not correspond to nitric oxide.

It should be noted that, in the strains belonging to the first two categories, the NO produced does not decrease at the end of growth and that it therefore does not appear to be reused by the propionibacteria: there is thus accumulation of NO.

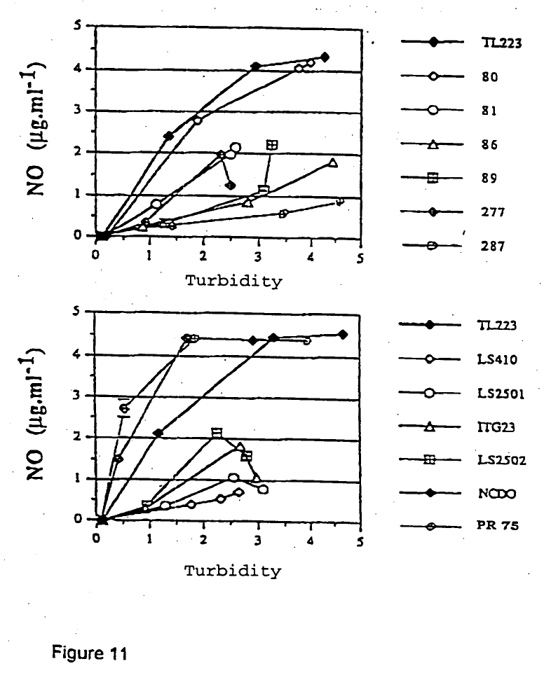

FIG. 11 represents the variations in NO production as a function of the turbidity for the 13 abovementioned propionibacterium strains, cultured on YEL medium in the presence of 550 μM nitrate. The strain TL223 was represented in each case for comparative purposes.

The vertical bars represent the +standard error of mean for n=3, when they are larger than the symbol.

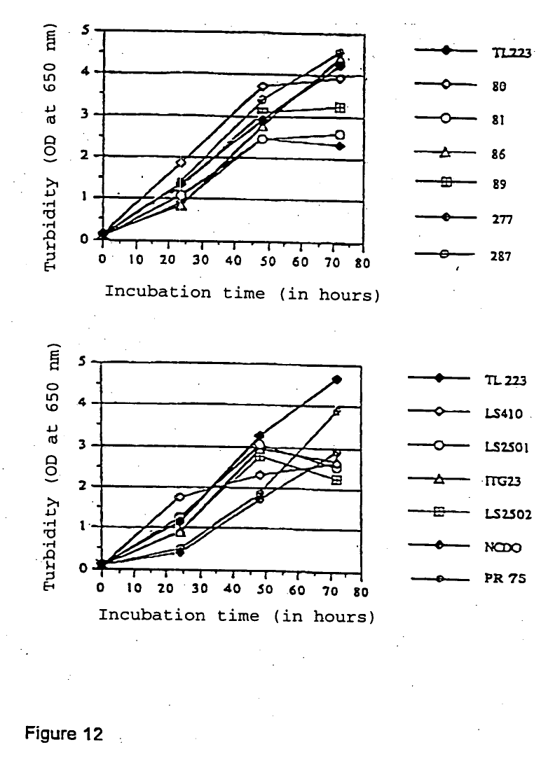

FIG. 12 represents the variations in the turbidity (OD at 650 nm) as a function of time for the 13 strains of propionibacterium analyzed, cultured on YEL medium in the presence of 550 μM nitrate.

The strain TL223 is represented in each case for comparative purposes.

The vertical bars represent the standard error of mean for n=3, when they are larger than the symbol.

The strains which produce the most NO (TL223, CNRZ80, NCDO1072 and PR75) all reach high turbidity values (4 to 5 OD units after incubation for 72 hours).

Among these strains, it should be noted that the bacteria NCDO1072 and PR75 grow less quickly than TL223, but are capable of accumulating large concentrations of NO very rapidly.

Thus, PR75 produces 2.8 μg of NO/ml for a turbidity of 0.5 OD units. The maximum NO production is recorded for turbidity values of about 1.5 OD units for NCDO1072 and PR75, compared with 3.4 OD units for TL223.

In order to “refine” these results, the change in the NO, nitrate and nitrite concentrations of the culture media for the 13 strains of propionibacterium studied, as a function of the incubation time was analyzed.

The results obtained are collated in the table below, in which the concentrations are expressed in μM. For each strain, the measurements were taken on a tube, after removal of the bacteria by centrifugation.

| | Concentration | | | Concentration | | | Production of | | |
|---|---|---|---|---|---|---|---|---|---|
| | of NO₃ | | | of NO₂ | | | NO. | | |
| | 24 h | 48 h | 72 h | 24 h | 48 h | 72 h | 24 h | 48 h | 72h |
| P. freudenreichii | | | | | | | | | |
| LS410 | 674 | 638 | 596 | 0 | 5 | 22 | 13 | 18 | 23 |
| LS2501 | 645 | 649 | 406 | 6 | 13 | 215 | 12 | 26 | 35 |
| LS2502 | 338 | 0 | 0 | 176 | 122 | 16 | 12 | 53 | 72 |
| ITG23 | 441 | 218 | 24 | 11 | 246 | 278 | 10 | 36 | 62 |
| CNRZ89 | 595 | 160 | 42 | 16 | 171 | 154 | 10 | 38 | 74 |
| CNRZ277 | 559 | 435 | 246 | 17 | 3 | 85 | 12 | 42 | 66 |
| CNRZ81 | 0 | 0 | 0 | 400 | 12 | 0 | 26 | 66 | 71 |
| P. acidipropionici | | | | | | | | | |
| TL223 | 0 | 0 | 0 | 271 | 0 | 0 | 71 | 149 | 152 |
| NCDO1072 | 0 | 2 | 0 | 370 | 3 | 0 | 50 | 148 | 146 |
| PR75 | 11 | 2 | 0 | 185 | 1 | 0 | 91 | 147 | 146 |
| CNRZ80 | 0 | 0 | 0 | 146 | 0 | 0 | 93 | 136 | 140 |
| CNRZ86 | 594 | 491 | 0 | 14 | 41 | 405 | 9 | 28 | 62 |
| CNRZ287 | 624 | 570 | 646 | 0 | 0 | 5 | 9 | 20 | 29 |

This table allows the following observations to be made:
the strains producing 4 μg of NO/ml are capable of entirely reducing the available nitrate (i.e. 550 μM) after 24 hours of incubation. Furthermore, they can totally reduce the nitrite obtained during the first 48 hours of incubation,
the strains LS410 and CNRZ287, which produce very low levels of NO, are not capable of significantly absorbing the nitrate present in the medium,
for the strains which display an intermediate accumulation of NO, very different changes in the nitrate and nitrite concentrations can be observed, reflecting very different rates of absorption of NO₃⁻ and/or of reduction of NO₃⁻ and of NO₂⁻. Thus, the strain CNRZ81 is capable of reducing all of the nitrate after 24 hours of incubation. After 48 hours, CNRZ81 also reduces all of the NO₂⁻ obtained by reduction of the NO₃⁻. In contrast, CNRZ277 still contains 246 μM NO₃⁻ and 85 μM NO₂⁻ after 72 hours of incubation.

The tests summarized above revealed that certain propionibacterium strains are capable of reducing the nitrate in the culture medium and thus producing the nitrite required for the synthesis of NO.

It should be noted that the propionibacterium strains which produce the most NO (TL223, CNRZ80, NCDO1072 and PR75) all belong to the species P. acidipropionici and, moreover, all have nitrate reductase activity.

In contrast, the strains apparently producing less NO, or even none at all (detection limit of the mass spectrometer) are also bacteria which have no known nitrate reductase activity (LS410, LS2501 and CNRZ287).

For certain propionibacterium strains, it appears that the NO production kinetics are not directly linked to the nitrate reductase activity.

Given these results, the invention also relates to an absorbable dietary or medicinal composition, characterized in that it consists of a preparation containing a large amount, preferably more than 10⁹ cells/g of propionibacterium strains selected as a function of their capacity to release and/or accumulate nitric oxide in a proportion of at least 1 μg/ml of YEL medium containing 550 μM nitrate.

According to another characteristic of the invention, this composition contains propionibacteria belonging to at least one of the strains TL223, CNRZ80, CNRZ86 and NCDO1072 of the species P. acidipropionici.

Among these strains, the strain TL223 proved to be particularly advantageous.

It should also be noted that the strain CNRZ80 is of quite specific value from a point of view of “productivity” given that it is capable of accumulating a high concentration of NO, and of doing so very rapidly (for relatively low turbidity values).

According to another characteristic of the invention, this composition contains propionibacteria belonging to at least one of the strains ITG23, CNRZ81, CNRZ89, CNRZ2.77 and LS2502 of the species P. freudenreichii.

It should be noted that, in accordance with another characteristic of the invention, the composition can also contain other bacteria, such as bifidobacteria and or lactic acid bacteria.

In order to complete the results obtained above, tests were carried out on two types of non-propionic bacteria: E. coli and Lactobacillus farciminis, known for their ability to reduce nitrites.

The results of these tests are summarized below:

7—Study of the Production of NO by the Strains E. coli and L. farciminis

These complementary tests were mainly carried out given the existence of the publication “Heme-dependent and heme-independent nitrite reduction by lactic acid bacteria results in different N-containing products” Gudrun Wolf, Elke K. Arendt, Ute Pfähler and Walter P. Hammes—International Journal of Food Microbiology, 10 (1990) 323-330) which mentions that certain lactic acid bacteria (L. farciminis) are capable of producing nitric oxide from nitrite.

Preliminary experiments showed that after growing the strain L. farciminis for 5 h 30 min in MRS supplemented with 1 mM nitrate, nitrate and nitrite were no longer detected in the culture medium.

The same observation was made as regards the strain E. coli after growth for 7 h 30 min on BHI medium supplemented with 1 mM nitrate.

It is known that, during its growth, the strain L. farciminis acidifies the MRS medium (about pH 5 after culturing for 5-6 hours).

It was possible to observe, from tests carried out on YEL medium, that nitrites are converted into NO in acidic medium.

These tests were carried out under the following experimental conditions:
acidification of the YEL medium with HCl,
nitrite supplied at a concentration of 400 μM,
autoclaving of the media,
three repetitions.

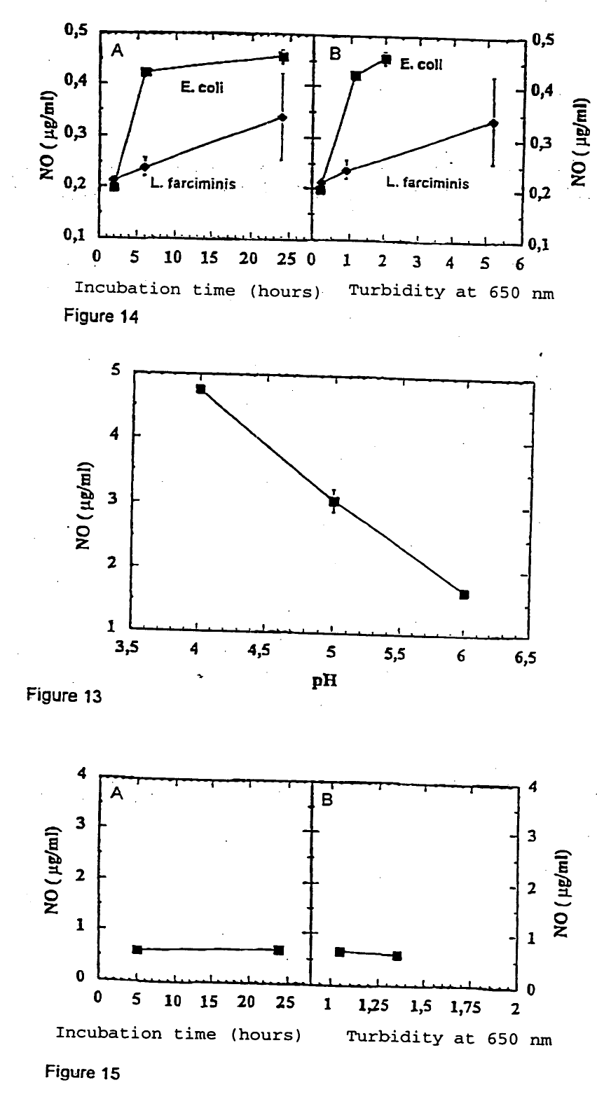

The results obtained are collated in FIG. 13 which represents the variations, as a function of the pH, in the production of NO in the YEL medium supplemented with nitrites after incubation at 37° C. for 24 hours.

This figure is able to prove that there is appreciable production of NO from the nitrite in the medium when this medium is acidic, this production increasing as the pH decreases.

Consequently, comparative tests of NO production by L. farciminis and by E. coli which is reputed not to produce NO (Brittain T, Blackmore R, Greenwood C & Thomson A J (1992)—Bacterial nitrite—reducing enzymes—Eur. J. Bio. Chem., 209, 793-802), were carried out.

These tests were carried out under the following conditions:
incubation at 37° C. on BHI medium (E. coli) or MRS medium (L. farciminis),
nitrate supplied to the medium at a concentration of 1 mM,
flushing of the atmosphere of each tube with helium for 100 seconds,
three repetitions,
measurement of the turbidity at the end of incubation.

These tests gave the results reported in FIG. 14.

More specifically:

FIG. 14A represents the variations in NO production as a function of the incubation time,

FIG. 14B represents the variations in NO production as a function of the turbidity of the medium.

It should be noted that the values obtained for the NO production are all markedly lower than the threshold of 1 μg/ml which was considered above as significant: the result of this is that there is no accumulation of NO in the culture tubes.

These results thus indicate that the absence of nitrate and nitrite observed in the preliminary experiment after 5 h 30 min (L. farciminis) and 7 h 30 min (E. coli), respectively, is not compensated for by an accumulation of NO which might be either of chemical origin (associated with acidification of the medium) or of bacterial origin.

In the case of the L. farciminis strain, these results were confirmed by tests carried out on bacteria in the form of resting cells at a pH adjusted to 6.5 by a phosphate buffer containing lactate, under the following operating conditions:
incubation at 37° C.,
nitrate supplied at a concentration of 400 μM,
flushing of the atmosphere of each tube with helium for 100 seconds
three repetitions,
measurement of the turbidity at the end of incubation.

This analysis gave the results reported in FIG. 15:

FIG. 15A represents the variations in NO production as a function of the incubation time,

FIG. 15B represents the variations in NO production as a function of the turbidity.

These results confirm those obtained above, i.e. that the amounts of NO produced are too small to be significant and thus that the L. farciminis strain is not capable of accumulating nitric oxide. However, it is possible that this strain produces nitric oxide at the start of growth, but that any NO produced is reused by the bacterium.

Complementary tests were carried out on the strains TL223 and CNRZ80 in the form of resting cells after incubation at 30 and also at 37° C.

8—Change in NO Production by Propionibacteria in the form of Resting Cells

This experiment was carried out under the following conditions:
resting cells suspended in a phosphate buffer containing lactate, at pH 6.5,
incubation at 30° C. or at 37° C.,
nitrite supplied at a concentration of 400 μM,
flushing of the atmosphere of each tube with helium for 100 seconds,
three repetitions,
measurement of the turbidity at the end of incubation.

It should be noted that, during the tests relating to the incubation at 30° C., in addition to the strains TL223 and CNRZ80, the strain CNRZ81 was examined with a twofold bacterial concentration.

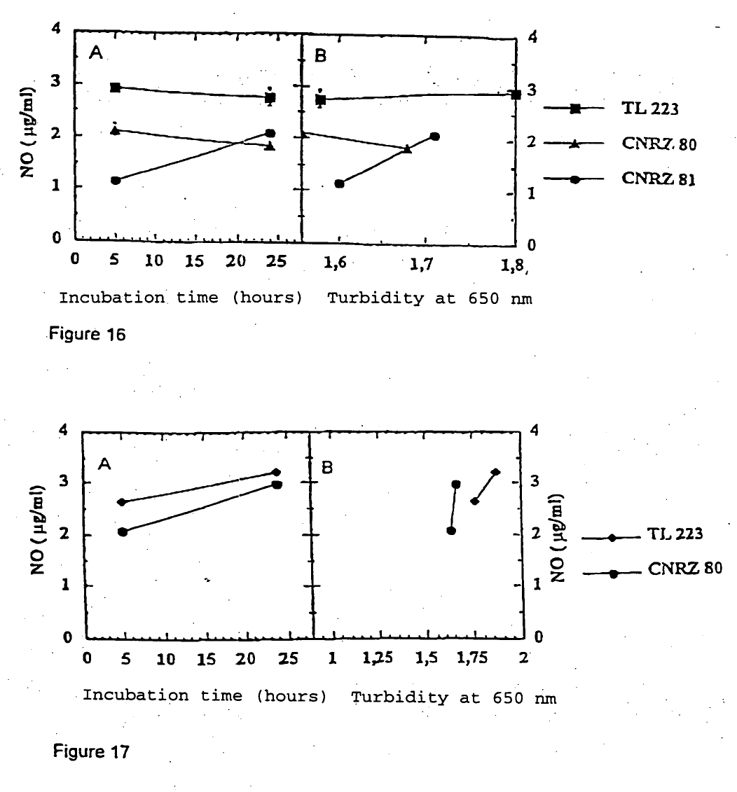

This experiment gave the results reported in FIGS. 16 and 17. More specifically:

FIG. 16A represents the variations in NO production by resting cells at 30° C. as a function of the incubation time,

FIG. 16B represents the variations in NO production by resting cells at 30° C. as a function of the turbidity of the medium,

FIG. 17A represents the variations in NO production by resting cells at 37° C. as a function of the incubation time,

FIG. 17B represents the variations in NO production by resting cells at 37° C. as a function of the turbidity of the medium.

These results are able to prove that there is a consequent production of NO by resting cells not only in the case of the two strains of the species P. acidipropionici (TL223 and CNRZ80), but also in the case of the strain of the species P. freudenreichii (CNRZ81). The strain TL223 is the most productive.

Globally, for identical bacterial concentrations, the NO production by resting cells is of the same order as that observed in the case of bacteria cultured on YEL medium.

The production of NO by resting cells occurs essentially during the first five hours of incubation; beyond this period, the production is low.

It was thus possible to observe that, at 37° C., the production of NO is identical (TL223) or slightly higher (CNRZ80) than that obtained at 30° C.

The advantages associated with the ingestion of propionibacteria were, in addition, confirmed by investigations performed in vivo on healthy humans.

9—Study of the Effect of Ingestion of Propionibacteria on Intestinal Transit in Healthy Humans

This study was carried out in a hospital environment at the University Hospital of Caen on a series of 19 healthy male volunteers.

At the start of this test, each volunteer was given 10 radio-opaque markers to absorb, for 8 consecutive days, in accordance with the procedure described in the publications Arhan P, Devroede G, Jehannin B et al. Dis Colon Rectum 1981; 24:625-9 and Bouchoucha M, Devroede G. Arhan P et al. Dis Colon. Rectum 1992; 35:773-82.

According to this procedure, study of the transit is carried out by counting the radio-opaque markers ingested in the different areas of the abdominal cavity, which are distributed on an anterior abdominal image. These areas (right colon, left colon and rectosigmoid) are defined by imaginary lines joining the 5th lumbar vertebra to the contour of the pelvic cavity. The transit time is calculated according to the formula. T=1/N n−Δt; N being equal to 10 markers, n representing the number of markers counted in a region and At being equal to 24 hours.

The day after this ingestion, i.e. the 9th day, the volunteers were made to undergo a radiography of the anterior abdomen without preparation.

Starting from the following day, i.e. the 10th day, each volunteer was given a gelatin capsule to ingest daily for 2 weeks, this capsule containing 5×10¹⁰ propionibacteria obtained from a bank of strains used in the cheesemaking industry, and thus entirely harmless to man.

A second study of the transit time similar to the first study was carried out during the second week of ingestion of the propionibacteria, i.e. from the 17th to the 26th day.

This study revealed a significant deceleration in the transit time of the left colon (p<0.05 in accordance with the Wilcoxon Matched-Paired Signed-Ranks statistical test); the transit times of the right colon and of the rectosigmoid were not significantly modified by the ingestion of propionibacteria.

This study thus proved that the ingestion of propionibacteria has an influence on intestinal motility; it can be assumed that these results are associated with the synthesis of nitric oxide by the propionibacteria.

1-12. (canceled)

13. A composition for use as an absorbable dietary supplement for human and animal consumption comprising more than 10⁹ cells propionibacteria per gram, said propionibacteria being capable of releasing at least 5 μg of nitric oxide after cultivating for at least 72 hours in 5 ml of a Yeast Extract Lactate medium containing at least 550 μmol/liter of nitrate or nitrite, and said propionibacteria being liable to release a physiologically significant amount of nitric oxide into the human and animal digestive tract for improving intestinal functions.

14. The composition according to claim 13, wherein said composition is added to a food product consisting of sources of dietary fibre, fermented milk, dessert cream, cake, and tonic drink.

15. A dietary supplement according to claim 13, wherein said supplement is a dehydrated or liquid preparation.

16. The composition according to claim 13, wherein said composition comprises further one or more bacteria selected from the group consisting of bifido bacteria and lactic acid bacteria.

17. Use of propionibacteria for improving intestinal functions comprising the steps of:
providing a supply of propionibacteria liable to release a physiologically significant amount of nitric oxide into the human and animal digestive tract;
adding more than 10⁹ cells of said propionibacteria per gram to an absorbable composition to form an absorbable dietary supplement for human and animal consumption; and
providing human or animal with said dietary supplement for consumption.

18. The use according to claim 17, wherein said propionibacteria being capable of releasing at least 5 μg of nitric oxide after cultivating for at least 72 hours in 5 ml of a Yeast Extract Lactate medium containing at least 550 μmol/liter of nitrate or nitrite.

19. The use according to claim 17, comprising the step of adding said dietary supplement to a food product.

20. The use according to claim 17, comprising further the step of adding one or more bacteria selected from the group consisting of bifido bacteria and lactic acid bacteria to the dietary supplement.
